(19) United States
(12) Patent Application Publication
(10) Pub. No.: US 2026/0061020 A1
(43) Pub. Date: Mar. 5, 2026

(54) BUTYRATE- & CURCUMINOID-CONTAINING COMPOSITIONS

(71) Applicant: Franco Cavaleri, Surrrey (CA)

(72) Inventor: Franco Cavaleri, Surrrey (CA)

(21) Appl. No.: 19/171,446

(22) Filed: Apr. 7, 2025

Related U.S. Application Data
(60) Provisional application No. 63/688,213, filed on Aug. 28, 2024.

(51) Int. Cl. A61K 36/9066; A61K 9/14; A61K 9/48; A61K 31/12; A61K 31/19

(52) U.S. Cl. CPC A61K 36/9066; A61K 9/14; A61K 9/48; A61K 31/12; A61K 31/19

(57) ABSTRACT

Compositions and methods for providing a person with an exogenous and therapeutically effective supply of short chain fatty acids and turmeric extracts (and/or curcuminoids) are disclosed. The compositions include purified butyrate and dried turmeric extracts. The compositions may optionally include purified butyrate and dried turmeric extracts spiked with additional curcumin I, curcumin II, and/or curcumin III. Alternatively, the compositions may include short chain fatty acids (such as butyrate) and purified curcumin I, curcumin II, and/or curcumin III.

### CROSS-REFERENCE TO RELATED APPLICATIONS

This application claims priority to U.S. provisional patent application Ser. No. 63/688,213, filed on Aug. 28, 2024.

### FIELD OF THE INVENTION

The field of the present invention relates to certain compositions that comprise a combination of short chain fatty acids (such as butyrate) and turmeric extracts—or, alternatively, short chain fatty acids (such as butyrate) and purified curcuminoids, which provide the various health benefits described herein.

### BACKGROUND OF THE INVENTION

There is an ongoing need within society for dietary and nutritional supplements that are effective to reduce inflammation, assist with weight loss, provide energy, help prevent heart disease, prevent and/or treat various cancers, and prevent and/or treat many other human diseases. Although there are currently-available pharmaceutical products that are specifically designed to address certain of such conditions, there is a continuing need for less expensive dietary and nutritional supplements that may be used and consumed to address the same needs.

As the following will demonstrate, the compositions and methods described herein are useful for providing an exogenous supply of short chain fatty acids and curcuminoids, which have been shown to provide a person with an effective means to reduce inflammation, assist with weight loss, and to help prevent heart and other human diseases.

### SUMMARY OF THE INVENTION

According to certain aspects of the invention, compositions are provided that include combinations of short chain fatty acids (particularly butyrate) and whole turmeric extracts. According to other aspects of the invention, compositions are provided that include combinations of short chain fatty acids (particularly butyrate) and whole turmeric extracts that include elevated levels of one or more curcuminoids (e.g., the whole turmeric extracts may be spiked with of one or more additional amounts of curcuminoids). According to still further aspects of the present invention, compositions are provided that include combinations of short chain fatty acids (particularly butyrate) and one or more purified curcuminoids.

The compositions of the present invention offer a multitude of benefits and can be used for numerous applications and indications. For example, oral formulations of such compositions may be used to provide a person with an effective means to reduce inflammation, to assist with weight loss, to help prevent heart disease, to induce thermogenic activity, to induce lipolysis, to induce brown adipose fat cell differentiation, and/or to prevent or treat various human diseases, such as cancer, neurodegenerative diseases, and other diseases.

The above-mentioned and additional features of the present invention are further illustrated in the Detailed Description contained herein.

### BRIEF DESCRIPTION OF THE FIGURES

FIG. 1 is a bar graph showing the anti-inflammatory effects (mRNA expression levels of TNF-α) of certain compositions of the present invention within HCT116 cells (inflammation was induced within the cells via TNF-α).

FIG. 2 is a bar graph showing the anti-inflammatory effects (mRNA expression levels of IL-8) of certain compositions of the present invention within HCT116 cells (inflammation was induced within the cells via TNF-α).

### DETAILED DESCRIPTION OF THE INVENTION

The following will describe, in detail, several preferred embodiments of the present invention. These embodiments are provided by way of explanation only, and thus, should not unduly restrict the scope of the invention. In fact, those of ordinary skill in the art will appreciate upon reading the present specification and viewing the present drawings that the invention teaches many variations and modifications, and that numerous variations of the invention may be employed, used and made without departing from the scope and spirit of the invention.

As mentioned above, the present invention includes three primary embodiments. First, according to certain preferred embodiments of the invention, compositions are provided that include combinations of short chain fatty acids (particularly butyrate) and whole turmeric extracts. Second, according to other preferred embodiments of the invention, compositions are provided that include combinations of short chain fatty acids (particularly butyrate) and whole turmeric extracts that include elevated levels of one or more curcuminoids (e.g., turmeric extracts may be spiked with additional amounts of curcumin I, curcumin II, and/or curcumin III). In certain examples, the dried turmeric extract component includes 50% (w/w) of bisdemethoxycurcumin (curcumin III), i.e., a whole turmeric extract is spiked with additional bisdemethoxycurcumin (curcumin III) to achieve a final concentration of at least 50% (w/w) bisdemethoxycurcumin (curcumin III) within such extract. Third, according to still further preferred embodiments of the present invention, compositions are provided that include combinations of short chain fatty acids (particularly butyrate) and one or more purified curcuminoids.

### Short Chain Fatty Acids

According to certain preferred embodiments of the present invention, compositions are provided that include one or more short chain fatty acids. In certain embodiments, the short chain fatty acids consist of purified butyric acid, butyrate salts, propionic acid, propionate salts, acetic acid, and/or acetate salts. In certain preferred embodiments, the short chain fatty acids consist of purified butyrate salts, namely, butyrate sodium salt, butyrate calcium salt, and/or butyrate magnesium salt. The invention provides that the one or more sources of short chain fatty acids (such as butyric acid, butyrate salts, propionic acid, propionate salts, acetic acid, or acetate salts) will preferably exist in a purified form, such as at least 75%, 80%, 85%, 90%, or 95% purified (i.e., free from other contaminants). Butyrate or butanoate ion, C₃H₇COO⁻, is the conjugate base of butyric acid. The chemical structure of butyric acid is reproduced below:

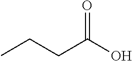

As described herein, the butyrate component of the compositions of the present invention will preferably exist in the form of salts, such as butyrate sodium salt, butyrate calcium salt, and/or butyrate magnesium salt.

### Turmeric Extracts & Curcuminoids

The compositions of the present invention further include plant-based sources of curcuminoids, namely, from the Indian spice turmeric (a member of Zingiberaceae). More particularly, the curcuminoid-related component of the present invention may include whole turmeric extracts; whole turmeric extracts that are spiked with additional curcumin I, curcumin II, and/or curcumin III; or purified forms of curcumin I, curcumin II, and/or curcumin III (and essentially free of other turmeric extract materials).

A typical curcumin extract comprises a mixture of curcumin I, desmethoxycurcumin (curcumin II), and bisdemethoxycurcumin (curcumin III). The term curcumin refers to the principal curcuminoid in the Indian spice turmeric plant. The IUPAC name for the curcumin I molecule is (1E,6E)-1,7-Bis(4-hydroxy-3-methoxyphenyl)-1,6-heptadiene-3,5-dione. Although curcumin I may exist in several different tautomeric forms, the enol form is illustrated below:

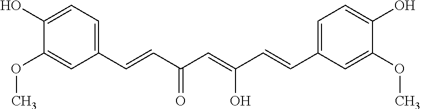 [SMILES: COc1cc(/C=C/C(=O)/C=C(O)/C=C/c2ccc(O)c(OC)c2)ccc1O]

The IUPAC name for the desmethoxycurcumin (curcumin II) molecule is (1E,6E)-1-(4-Hydroxy-3-methoxyphenyl)-7-(4-hydroxyphenyl) hepta-1,6-diene-3,5-dione, and has the chemical structure shown below:

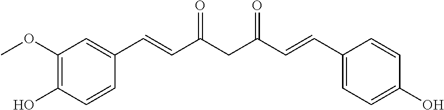 [SMILES: COc1cc(/C=C/C(=O)CC(=O)/C=C/c2ccc(O)cc2)ccc1O]

The IUPAC name for bisdemethoxycurcumin (curcumin III) that is used in the compositions and methods of the present invention is (1E,6E)-1,7-bis(4-hydroxyphenyl) hepta-1,6-diene-3,5-dione, and has the chemical structure shown below:

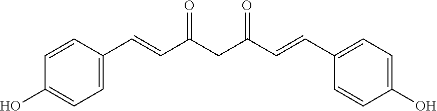 [SMILES: O=C(/C=C/c1ccc(O)cc1)CC(=O)/C=C/c1ccc(O)cc1]

According to certain preferred embodiments, the invention provides that the turmeric extracts described herein are obtained from a turmeric plant rhizome (Curcuma longa) and subsequently dried and concentrated to the desired levels. Alternatively, the invention provides that such extracts may be spiked with additional purified curcumin I, curcumin II, and/or curcumin III. Still further, according to other embodiments, such curcumin I, curcumin II, and/or curcumin Ill molecules may be used alone in purified form (or chemically synthesized) and used to formulate a composition described herein, such curcuminoid(s) may be at least 75%, 80%, 85%, 90%, or 95% purified (i.e., free from other contaminants).

The invention provides that the compositions may further include other pharmacologically active agents, such as acetyl-L-carnitine, R-alpha lipoic acid, green tea extract, vitamins, and various combinations of such agents. According to additional embodiments of the present invention, the compositions may (optionally) further include one or more ketones (or precursors thereof), such as purified beta-hydroxybutyrate or esters or propionate salts thereof. Non-limiting examples of such purified beta-hydroxybutyrate salts include a beta-hydroxybutyrate sodium salt, a beta-hydroxybutyrate calcium salt, and/or a beta-hydroxybutyrate magnesium salt.

In certain embodiments, the present invention further includes methods for reducing inflammation, assisting with weight loss, and/or helping to prevent heart disease. In addition, the present invention further includes methods for reducing the expression of pro-inflammatory proteins, such as TNF-α (tumor necrosis factor-alpha) and IL-8 (interleukin 8). Still further, the present invention includes methods for inducing thermogenic activity, inducing lipolysis, and/or to inducing brown adipose fat cell differentiation in a subject. Such methods generally include orally administering one of the pharmacologic compositions described herein to a subject.

In such embodiments, the methods and compositions are preferably effective to deliver at least 100 mg of short chain fatty acids (such as purified butyrate) and 100 mg of dried turmeric extract. Such amounts may be administered within a single dose—or, alternatively, through a multiple dose/daily regime. In other embodiments, the methods and compositions are effective to deliver at least 200 mg, 300 mg, 400 mg, 500 mg, 600 mg, 700 mg, 800 mg, or 900 mg of short chain fatty acid (such as butyrate) and at least 200 mg, 300 mg, 400 mg, 500 mg, 600 mg, 700 mg, 800 mg, or 900 mg of dried turmeric extract, purified curcuminoids, or derivatives of such purified curcuminoids. As described further below in the Examples, such compositions are preferably delivered to a person in the form of oral capsules or dry powders.

Notwithstanding the preferred embodiments and Examples described herein, the invention provides that the compositions of the present invention may be administered in any desired and effective manner, e.g., as pharmaceutical compositions or nutritional supplements for oral ingestion. More particularly, for example, pharmaceutically acceptable compositions or nutritional supplements of the invention may comprise one or more of the compositions described herein with one or more acceptable carriers. Regardless of the route of administration selected, the compositions may be formulated into acceptable dosage forms by conventional methods known to those of skill in the art. For example, acceptable carriers include, but are not limited to, sugars (e.g., lactose, sucrose, mannitol, and sorbitol), silicon dioxide, starches, cellulose preparations (such as microcrystalline cellulose), calcium phosphates (e.g., dicalcium phosphate, tricalcium phosphate and calcium hydrogen phosphate), sodium citrate, water, aqueous solutions, alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes, paraffins, silicones, talc, silicylate, etc.

Each acceptable carrier used in a pharmaceutical composition or nutritional supplement of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art.

The pharmaceutical compositions and nutritional supplements of the invention may, optionally, contain additional ingredients and/or materials commonly used in pharmaceutical compositions and/or nutritional supplements. These ingredients and materials include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxy methyl cellulose and sodium carbonate; (5) solution retarding agents, such as paraffin; (6) absorption accelerators, such as quaternary ammonium compounds; (7) wetting agents, such as cetyl alcohol and glycerol monosterate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; (28) vitamins and minerals; (29) proteins that carry therapeutic or nutritional benefits, such as whey protein and other milk-derived proteins; and (30) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Compositions and nutritional supplements suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, or a paste. These formulations may be prepared by methods known in the art, e.g., by means of conventional pan-coating, mixing, granulation or lyophilization processes.

Solid dosage forms for oral administration (capsules, tablets, pills, powders, granules and the like) may be prepared by mixing the active ingredient(s) with one or more acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as capsules, pills and granules, may optionally be scored or prepared with coatings and shells, such as enteric coatings and other coatings well known in the art. The tablets, and other solid dosage forms, may also be formulated so as to provide slow or controlled release of the active ingredient therein. They may be sterilized by, for example, filtration through a bacteria-retaining filter. These compositions may also optionally contain opacifying agents that release the active ingredient only, or preferentially, in a certain portion of the gastrointestinal tract, optionally, in a delayed manner. The active ingredient can also be in a microencapsulated form.

Liquid dosage forms for oral administration include acceptable emulsions, microemulsions, solutions, suspensions, syrups, and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

### EXAMPLES

The following Examples describe various compositions and methods of the present invention, which include butyrate salts (and/or esters or propionate salts thereof), in combination with turmeric extracts (and/or purified curcuminoids). The invention provides that additional optimizing ingredients may be included in the formulations exemplified below. For example, acetyl carnitine may be included, to provide a fatty acid transport mechanism facilitator. In addition, R-alpha lipoic acid may be included to improve insulin efficacy and to drive serum glucose levels down to be conducive to ketogenesis and ketone body prevalence as an ATP substrate.

### Example 1—Capsules

| Component | Amount/Capsule (mg) |
|---|---|
| Dried Turmeric Extract | 275 |
| Butyrate Sodium Salt; | 220 |
| Butyrate Calcium Salt; and/or | |
| Butyrate Magnesium Salt | |
| Total Per Capsule(s) | 495 |

### Example 2—Capsules

| Component | Amount/Capsule (mg) |
|---|---|
| Purified Curcumin I, II, and/or III | 300 |
| Butyrate Sodium Salt; | 200 |
| Butyrate Calcium Salt; and/or | |
| Butyrate Magnesium Salt | |
| Total Per Capsule(s) | 500 |

### Example 3—Dried Powder

| Component | Amount (mg) |
|---|---|
| Dried Turmeric Extract | 300 |
| Butyrate Sodium Salt; | 300 |
| Butyrate Calcium Salt; and/or | |
| Butyrate Magnesium Salt | |
| Total Per Capsule(s) | 600 |

### Example 4—Dried Powder

| Component | Amount (mg) |
|---|---|
| Purified Curcumin I, II, and/or III | 310 |
| Butyrate Sodium Salt; | 310 |
| Butyrate Calcium Salt; and/or | |
| Butyrate Magnesium Salt | |
| Total Per Capsule(s) | 620 |

### Example 5—Reduction of TNF-α and IL-8 Gene Expression

In this Example, the anti-inflammatory effects of a combination of (1) sodium butyrate and (2) a turmeric extract that included approximately 50% (w/w) of bisdemethoxycurcumin (curcumin III) was tested. More particularly, the ability of a combination of (1) sodium butyrate and (2) a turmeric extract that included approximately 50% (w/w) of bisdemethoxycurcumin (curcumin III) to reduce the expression levels of the pro-inflammatory proteins, TNF-α and IL-8, was tested in HCT116 cells (a cellular model for intestinal epithelial cells).

In this Example, prior to treating the HCT116 cells with the test compositions, the cell media was replaced with 5% FBS-containing media to optimize cell viability and ensure reproducibility. A first group of cells were then treated with 10 μg/ml of BDMC50 (a turmeric extract that included elevated amounts of bisdemethoxycurcumin, i.e., approximately 50% (w/w) of bisdemethoxycurcumin) for 24 hours, and a second group of cells were treated with 5 mM sodium butyrate+BDMC50 for 24 hours. After the 24-hour incubation period, inflammation was induced in the HCT116 cells by application of TNF-α at 10 ng/ml for 4 hours. The cells were then collected for further analysis. More particularly, the expression levels of two key pro-inflammatory markers (TNF-α and IL-8) were measured in the collected cells via qRT-PCR analysis.

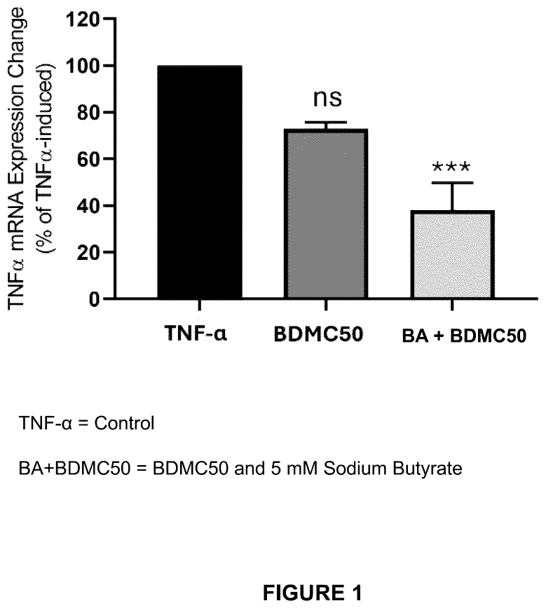

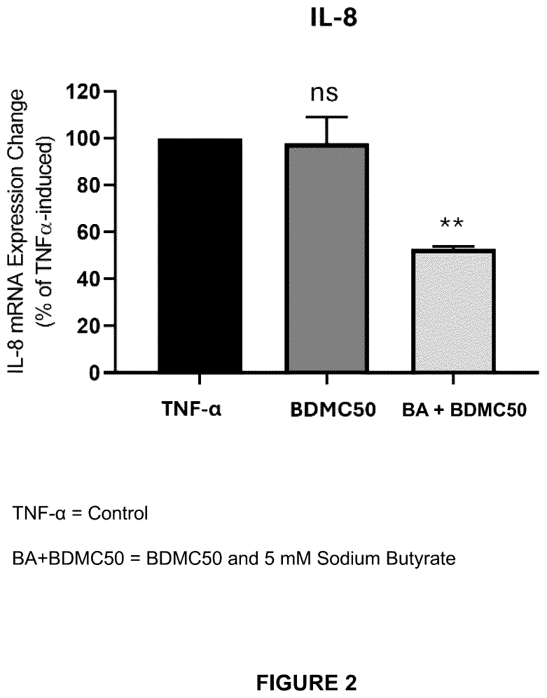

As shown in FIGS. 1 and 2, the cells treated with 5 mM sodium butyrate+BDMC50 exhibited a much greater suppression of pro-inflammatory gene expression (TNF-α and IL-8) compared to BDMC50 alone or whole curcumin extract (not shown). The invention provides that this observed synergistic activity stems from the combined composition's ability to regulate inflammation through different but complementary mechanisms—sodium butyrate, as a histone deacetylase inhibitor, enhances the expression of anti-inflammatory markers, thereby amplifying the effects of the BDMC50 component (which includes curcuminoids that primarily inhibit the NF-κB pathway, a central regulator of inflammation, at a downstream level).

The many aspects and benefits of the invention are apparent from the detailed description, and thus, it is intended for the following claims to cover all such aspects and benefits of the invention that fall within the scope and spirit of the invention. In addition, because numerous modifications and variations will be obvious and readily occur to those skilled in the art, the claims should not be construed to limit the invention to the exact construction and operation illustrated and described herein. Accordingly, all suitable modifications and equivalents should be understood to fall within the scope of the invention as claimed herein.

What is claimed is:

1. A composition comprising:
(a) purified butyrate or salts thereof, wherein the purified butyrate includes a butyrate sodium salt, a butyrate calcium salt, and/or a butyrate magnesium salt; and
(b) dried turmeric extract.

2. A composition comprising:
(a) purified butyrate or salts thereof, wherein the purified butyrate includes a butyrate sodium salt, a butyrate calcium salt, and/or a butyrate magnesium salt; and
(b) dried turmeric extract, wherein the dried turmeric extract contains elevated levels of curcumin I, curcumin II, and/or curcumin III.

3. The composition of claim 2, wherein the dried turmeric extract includes at least 50% (w/w) of bisdemethoxycurcumin (curcumin III).

4. A composition comprising:
(a) purified butyrate or salts thereof, wherein the purified butyrate includes a butyrate sodium salt, a butyrate calcium salt, and/or a butyrate magnesium salt; and
(b) purified curcumin I, curcumin II, and/or curcumin III.

5. A method for reducing inflammation in a group of cells that comprises administering to the cells an effective amount of the composition of claim 1.

6. A method for reducing inflammation in a group of cells that comprises administering to the cells an effective amount of the composition of claim 2.

7. A method for reducing inflammation in a group of cells that comprises administering to the cells an effective amount of the composition of claim 3.

8. A method for reducing inflammation in a group of cells that comprises administering to the cells an effective amount of the composition of claim 4.

9. A method for reducing expression of TNF-α in a group of cells that comprises administering to the cells an effective amount of the composition of claim 1.

10. A method for reducing expression of TNF-α in a group of cells that comprises administering to the cells an effective amount of the composition of claim 2.

11. A method for reducing expression of TNF-α in a group of cells that comprises administering to the cells an effective amount of the composition of claim 3.

12. A method for reducing expression of TNF-α in a group of cells that comprises administering to the cells an effective amount of the composition of claim 4.

13. A method for reducing expression of IL-8 in a group of cells that comprises administering to the cells an effective amount of the composition of claim 1.

14. A method for reducing expression of IL-8 in a group of cells that comprises administering to the cells an effective amount of the composition of claim 2.

15. A method for reducing expression of IL-8 in a group of cells that comprises administering to the cells an effective amount of the composition of claim 3.

16. A method for reducing expression of IL-8 in a group of cells that comprises administering to the cells an effective amount of the composition of claim 4.
